(19) United States
(12) Patent Application Publication
(10) Pub. No.: US 2020/0093385 A1
(43) Pub. Date: Mar. 26, 2020

(54) HEATED BLOOD PRESSURE CUFF ASSEMBLY

(71) Applicant: Angela Felker, Cumming, GA (US)

(72) Inventor: Angela Felker, Cumming, GA (US)

(21) Appl. No.: 16/136,332

(22) Filed: Sep. 20, 2018

(51) Int. Cl. A61B 5/022; A61B 5/00; A61F 7/02

(52) U.S. Cl. CPC A61B 5/02233; A61B 5/742; A61F 2007/0282; A61B 2562/225; A61F 2007/0228; A61F 7/02

(57) ABSTRACT

A heated blood pressure cuff assembly for warming a blood pressure cuff includes an electronic blood pressure monitor that has a plurality of controls and a display. A blood pressure cuff is provided and is in fluid communication with the electronic blood pressure monitor for checking blood pressure in a patient. A heating unit is coupled to the blood pressure cuff and the heating unit is in thermal communication with the blood pressure cuff. The heating unit warms the blood pressure cuff when the heating unit is turned on thereby inhibiting a physiological response to cold in the patient when the blood pressure cuff is applied.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to heated cuff assemblies and more particularly pertains to a new heated cuff assembly for warming a blood pressure cuff.

### BRIEF SUMMARY OF THE INVENTION

An embodiment of the disclosure meets the needs presented above by generally comprising an electronic blood pressure monitor that has a plurality of controls and a display. A blood pressure cuff is provided and is in fluid communication with the electronic blood pressure monitor for checking blood pressure in a patient. A heating unit is coupled to the blood pressure cuff and the heating unit is in thermal communication with the blood pressure cuff. The heating unit warms the blood pressure cuff when the heating unit is turned on thereby inhibiting a physiological response to cold in the patient when the blood pressure cuff is applied.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a heated blood pressure cuff assembly according to an embodiment of the disclosure.

FIG. 2 is a front perspective view of an embodiment of the disclosure.

FIG. 3 is a cross sectional view taken along line 3-3 of FIG. 2 of an embodiment of the disclosure.

FIG. 4 is a back phantom view of an embodiment of the disclosure.

### DETAILED DESCRIPTION OF THE INVENTION

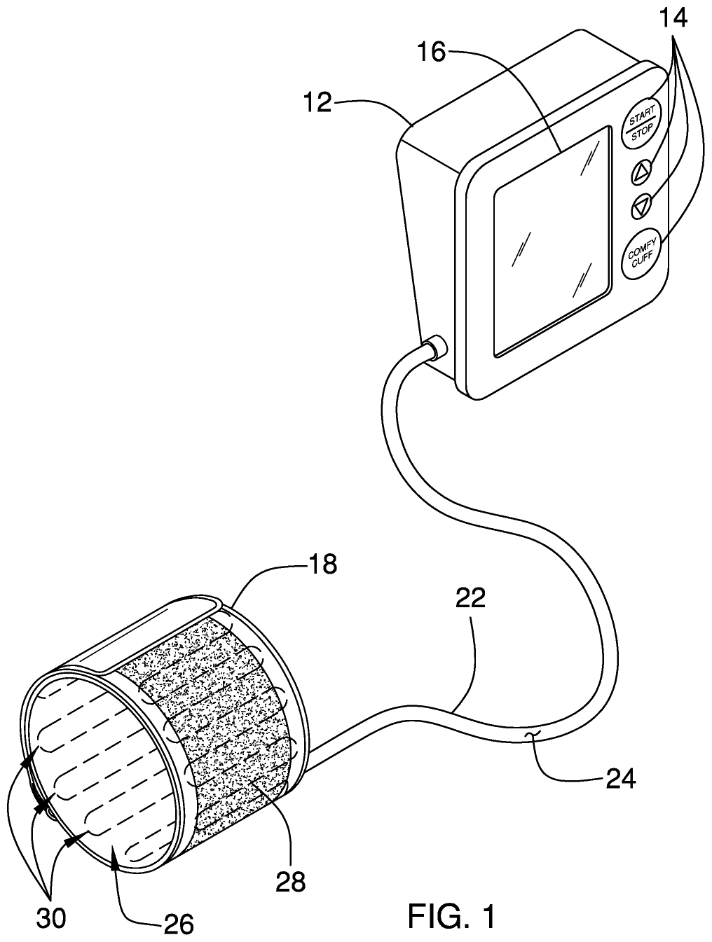

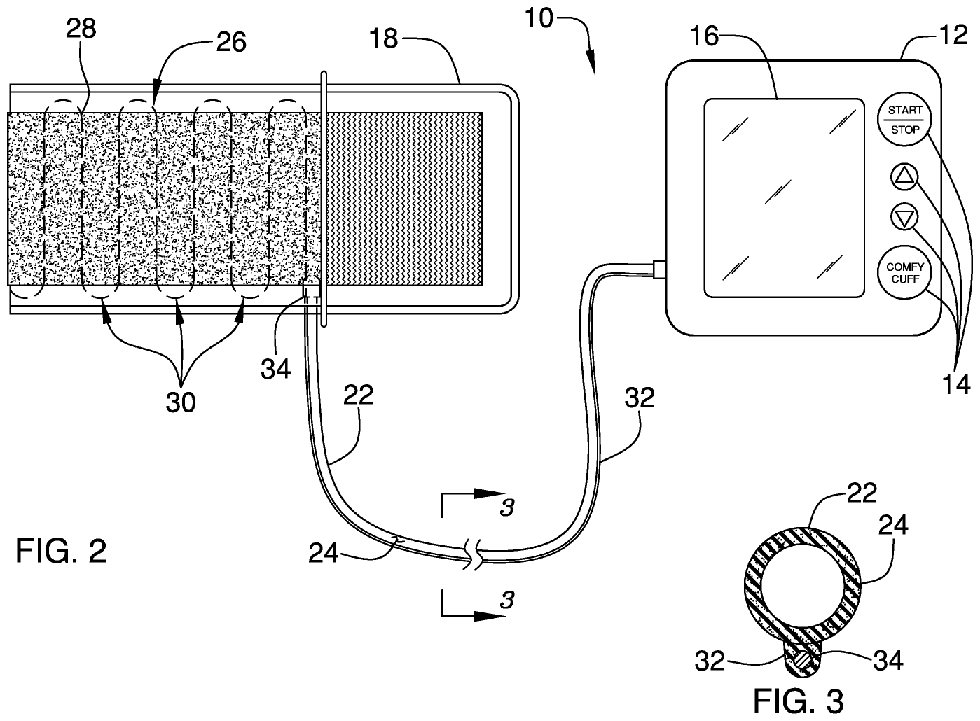

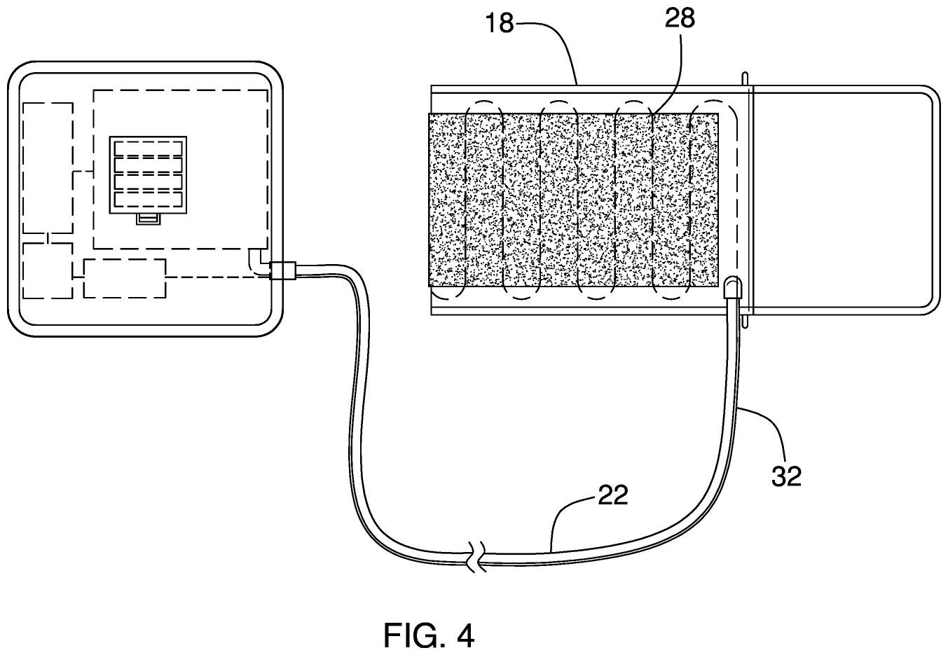

With reference now to the drawings, and in particular to FIGS. 1 through 4 thereof, a new heated cuff assembly embodying the principles and concepts of an embodiment of the disclosure and generally designated by the reference numeral 10 will be described.

As best illustrated in FIGS. 1 through 4, the heated blood pressure cuff assembly 10 generally comprises an electronic blood pressure monitor 12 that has a plurality of controls 14 and a display 16. The electronic blood pressure monitor 12 may be employed in a clinical setting, such as a hospital or a clinic. A blood pressure cuff 18 is included and the blood pressure cuff 18 is in fluid communication with the electronic blood pressure monitor 12 for checking blood pressure in a patient. The blood pressure cuff 18 includes a hose 22 that is fluidly coupled between the blood pressure cuff 18 and the electronic blood pressure monitor 12. The hose 22 has an outer surface 24 and the blood pressure cuff 18 may be of any conventional design commonly employed by health care professionals.

A heating unit 26 is provided and the heating unit 26 is coupled to the blood pressure cuff 18. The heating unit 26 is in thermal communication with the blood pressure cuff 18 such that the heating unit 26 warms the blood pressure cuff 18 when the heating unit 26 is turned on. Thus, the heating unit 26 inhibits a physiological response to cold in the patient when the blood pressure cuff 18 is applied. In this way the heating unit 26 ensures a more accurate blood pressure reading by avoiding increased heart rate, drop in blood pressure and other physiological responses to a cold touch.

The heating unit 26 comprises a heating element 28 that is positioned within the blood pressure cuff 18 and the heating element 28 is in thermal communication with the blood pressure cuff 18. The heating element 28 may be arranged to form a plurality of coils 30 within the blood pressure cuff 18. Additionally, the heating element 28 may be an electric heating element 28 with an operational temperature ranging between approximately 80.0 degrees Fahrenheit and 90.0 degrees Fahrenheit.

A tube 32 is coupled to the outer surface 24 of the hose 22 and the tube 32 is coextensive with the hose 22. A conductor 34 is positioned within the tube 32 and the conductor 34 is electrically coupled between the heating element 28 and the electronic blood pressure monitor 12. Thus, the heating element 28 is in electrical communication with the controls 14 on the electronic blood pressure monitor 12 for controlling operational parameters of the heating element 28. The controls 14 on the electronic blood pressure monitor 12 may include a power button for the heating element 28, a temperature increase button and a temperature decrease button, and the power button may have “comfy cuff” printed thereon.

In use, the heating element 28 is turned on prior to applying the blood pressure cuff 18 to the patient. Thus, the heating element 28 warms the blood pressure cuff 18 to a temperature that is near the body temperature of the patient. In this way the patient does not display any of the physiological responses to a cold touch, thereby enhancing accuracy of the blood pressure reading. Additionally, the heating element 28 serves to enhance comfort for the patient while the blood pressure is being checked.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heated blood pressure cuff assembly being selectively heated wherein said assembly is configured to inhibit a cold shock reaction in a patient, said assembly comprising:
an electronic blood pressure monitor having a plurality of controls and a display;
a blood pressure cuff being in fluid communication with said electronic blood pressure monitor for checking blood pressure in a patient; and
a heating unit being coupled to said blood pressure cuff, said heating unit being in thermal communication with said blood pressure cuff such that said heating unit warms said blood pressure cuff thereby inhibiting a physiological response to cold in the patient when said blood pressure cuff is applied.

2. The assembly according to claim 1, wherein said heating unit comprises a heating element being positioned within said blood pressure cuff, said heating element being in thermal communication with said blood pressure cuff.

3. The assembly according to claim 1, wherein:
said blood pressure cuff includes a hose having an outer surface; and
said heating unit includes a tube being coupled to said outer surface of said hose, said tube being coextensive with said hose.

4. The assembly according to claim 3, wherein said heating unit includes:
a heating element; and
a conductor being electrically coupled between said heating element and said electronic blood pressure monitor, said heating element being in electrical communication with said controls on said electronic blood pressure monitor for controlling operational parameters of said heating element.

5. A heated blood pressure cuff assembly being selectively heated wherein said assembly is configured to inhibit a cold shock reaction in a patient, said assembly comprising:
an electronic blood pressure monitor having a plurality of controls and a display;
a blood pressure cuff being in fluid communication with said electronic blood pressure monitor for checking blood pressure in a patient, said blood pressure cuff including a hose being fluidly coupled between said blood pressure cuff and said electronic blood pressure monitor, said hose having an outer surface; and
a heating unit being coupled to said blood pressure cuff, said heating unit being in thermal communication with said blood pressure cuff such that said heating unit warms said blood pressure cuff thereby inhibiting a physiological response to cold in the patient when said blood pressure cuff is applied, said heating unit comprising:
a heating element being positioned within said blood pressure cuff, said heating element being in thermal communication with said blood pressure cuff;
a tube being coupled to said outer surface of said hose, said tube being coextensive with said hose; and
a conductor being positioned within said tube, said conductor being electrically coupled between said heating element and said electronic blood pressure monitor, said heating element being in electrical communication with said controls on said electronic blood pressure monitor for controlling operational parameters of said heating element.
